(19) United States
(12) Patent Application Publication
(10) Pub. No.: US 2017/0231837 A1
(43) Pub. Date: Aug. 17, 2017

(54) Disposable Diaper And Cushion Sheet

(71) Applicant: OJI HOLDINGS CORPORATION, Tokyo (JP)

(72) Inventor: Izumi TASHIRO, Chuo-ku, Tokyo (JP)

(21) Appl. No.: 15/504,656

(22) PCT Filed: Aug. 5, 2015
(86) PCT No.: PCT/JP2015/072166

§ 371 Date: Feb. 17, 2017

(30) Foreign Application Priority Data
Aug. 20, 2014 (JP) 2014-167492

(51) Int. Cl. A61F 13/49; A61F 13/496; A61F 13/56; A61F 13/514

(52) U.S. Cl. CPC A61F 13/49011; A61F 13/51498; A61F 13/496; A61F 13/5633

(57) ABSTRACT

To improve the ventilation properties of a sheet used in a flute structure. This disposable diaper has formed therein, on at least one of a front part and a rear part, a cushion part (40). In the cushion part (40), in a state where a waist expansion member (43) is stretched in the width direction of the diaper, the member is sandwiched and secured between an inner sheet (41) and an outer sheet (42). In the cushion part (40), a cushion forming sheet (44) overlaps one or both of the inner sheet (41) and the outer sheet (42), and is intermittently joined thereto so that in the width direction joining portions (51) and nonjoining portions (52) are alternatingly continuous. Formed in the cushion forming sheet (44) are a plurality of openings (60) or depressions that are grouped into row groups (R) and column groups (C). A continuous line (A) links the plurality of openings (60) or depressions which belong to the column group (C), and the line is inclined at a prescribed angle (θ) with respect to a reference line (F) that extends in the longitudinal direction.

### TECHNICAL FIELD

The present invention relates to a disposable diaper. More specifically, the invention relates to a disposable diaper in which a waist gather having a high cushioning property may be formed in a waist end part of the diaper.

### BACKGROUND ART

A disposable diaper for an infant and a toddler or for an aged person has been known. Various types of disposable diapers are present. For example, a pants-type disposable diaper and a tape-type disposable diaper are known (for example, Patent Literature 1, Patent Literature 2, and Patent Literature 3).

The pants-type disposable diaper is a disposable diaper in which both side portions corresponding to a front body part and a rear body part are joined to each other in advance. Examples of the known pants-type disposable diaper include an integrated disposable diaper (Patent Literature 1) in which an outer packaging body on which an absorbent main body is placed is one body across a rear body part from a front body part, and a separation-type disposable diaper (Patent Literature 2) in which an outer packaging body is separated into a front body part and a rear body part, and an absorbent main body is connected between a front body part outer packaging body and a rear body part outer packaging body such that the absorbent main body is bridged therebetween. In addition, the tape-type disposable diaper is a disposable diaper in which an adhesive tape is attached to both side portions corresponding to a front body part and a rear body part, and the front body part and the rear body part are attached to each other using the adhesive tape at the time of wearing (Patent Literature 3).

In the pants-type disposable diaper, in general, a plurality of stretchable members is fixed in an extended state to a rim of a waist opening formed by attaching the front body part to the rear body part. As these stretchable members tighten the waist circumference of the wearer, the diaper may be worn without slipping down due to movement of the wearer or a weight of excreta. A function for preventing the diaper from slipping down by providing the stretchable members at the rim of the waist opening is important.

Meanwhile, when the diaper is excessively strongly fastened around the waist, the wearer may feel strongly tightened. In addition, when the diaper is strongly fastened, adhesion increases. In contrast, air permeability is degraded.

Herein, there has been a known technology for forming a waist gather to assign an appropriate tightening force to the circumference of the waist of the disposable diaper. In more detail, by fixing a plurality of stretchable members in an extended state between cover sheets included in the circumference of the waist of the disposable diaper, a wrinkle is generated in a cover sheet adjacent to a waist opening when these stretchable members contract. Due to the wrinkle, a waist gather is formed around the waist opening. This waist gather may improve adhesion of the disposable diaper by coming into contact of a skin of the wearer, and thus a fitting feeling around the waist is improved. In addition, the waist gather functions as a cushion for relieving the fastening being done to the wearer due to the stretchable members.

Further, Patent Literature 4 discloses a technology for providing a disposable diaper that has flexibility and a cushioning property in a whole waist gather. This technology related to the waist gather is also referred to as a “flute structure”. The flute structure includes a pair of sheets for fixing a waist stretchable member by interposing the waist stretchable member therebetween, and a sheet for forming a wrinkle (cushion forming sheet) joined to a top of the sheets by intermittent joining portions. In the flute structure, when the waist stretchable member contracts, the cushion forming sheet rises at a portion (non-joining portion) which is not joined. In this way, when a ventilation passage (space) is formed between the cushion forming sheet and the pair of sheets interposing the waist stretchable member therebetween, hardness of the pair of sheets interposing the waist stretchable member therebetween is unlikely to be directly delivered to the skin. For this reason, air permeability, flexibility, and a cushioning property may be assigned to the whole waist gather by forming the flute structure.

In addition, Patent Literature 5 discloses an invention for forming a plurality of tubular portions (ventilation passages) around a belly of a disposable diaper, and intermittently forming a plurality of apertures in a sheet member included in the tubular portions. In the invention of Patent Literature 5, the plurality of apertures is provided in a row along a projection of a sheet included in the tubular portions. For this reason, the plurality of apertures is linearly arranged in a direction in which the tubular portions extend (longitudinal direction of the disposable diaper). In this way, when the plurality of apertures is formed in the tubular portions around the belly of the disposable diaper, it is possible to enhance air permeability of a waist gather, and to soften a touch.

### CITATION LIST

### Patent Literature

Patent Literature 1: JP 2009-254662 A

Patent Literature 2: JP 2010-233733 A

Patent Literature 3: JP 2005-323685 A

Patent Literature 4: JP 2011-254996 A

Patent Literature 5: JP 2011-078477 A

### SUMMARY OF INVENTION

### Technical Problem

Incidentally, it is considered to use a sheet member in which the plurality of apertures disclosed in Patent Literature 5 is formed or a sheet member in which a plurality of cavities is formed in order to improve a touch and air permeability of the “flute structure” disclosed in Patent Literature 4.

However, as described in the foregoing, the plurality of apertures is linearly arranged in the longitudinal direction of the disposable diaper in the invention of Patent Literature 5. Then, a non-aperture region in which no aperture is formed in the longitudinal direction is present between rows of apertures in the invention of Patent Literature 5. That is, an aperture region and the non-aperture region are alternately present when viewed in a width direction of the disposable diaper in the invention of Patent Literature 5. In this way, when the aperture region and the non-aperture region are clearly distinguished in the sheet member for forming the tubular portion (ventilation passage), unevenness (deviation) in strength is generated in the sheet member. That is, in the aperture region of the sheet member, a plurality of apertures is formed in a row, and thus strength is relatively weak. In contrast, in the non-aperture region of the sheet member, no aperture is formed in the longitudinal direction, and thus strength is relatively strong. Therefore, in the present invention, a portion having weak strength and a portion having strong strength are alternately present in the width direction of the disposable diaper (stretching direction of the waist stretchable member). As a result, when apertures are formed as in Patent Literature 5, and when the waist stretchable member contracts, this contractile force is unlikely to be uniformly delivered to the whole sheet, and there is a problem that space inside the tubular portion (ventilation passage) is unlikely to efficiently inflates. In particular, in the invention described in Patent Literature 5, a plurality of apertures is formed only in a region along a vertex (projection) of the tubular portion. When apertures are formed only at the vertex of the tubular portion in this way, strength at a portion around the vertex of the tubular portion decreases, and thus space inside the tubular portion is unlikely to inflate.

Further, in the invention of Patent Literature 5, the plurality of apertures is formed only in the region along the vertex (projection) of the tubular portion. For this reason, in the invention of Patent Literature 5, no aperture is formed around the joining portion of the sheet member present at a root of the tubular portion. However, a portion around the joining portion of the sheet member easily becomes stuffy. For this reason, a configuration of Patent Literature 5 has a problem that stuffiness occurring around the joining portion of the sheet member may not be solved.

It is preferable that strength in the width direction of the whole sheet (stretching direction of the waist stretchable member) be as uniform as possible when the sheet member in which the plurality of apertures or cavities is formed is used in the “flute structure” of the disposable diaper as described above. In addition, it is preferable to ensure air permeability around the joining portion of the sheet member. In this regard, an object of the invention is to provide a technology capable of uniformizing strength of the sheet member employed in the “flute structure”, and improving air permeability around the joining portion.

### Solution to Problem

As a result of a keen examination of means for solving a conventional problem, the inventors of the invention have determined to form a plurality of apertures or cavities in a sheet member (cushion forming sheet) in an outermost layer of the above-described “flute structure”, and incline a column of the aperture or cavities at a predetermined angle with respect to a longitudinal direction of a diaper. In more detail, in the invention, the plurality of apertures or cavities formed in the sheet member are not arranged on a straight line in the longitudinal direction of the diaper, and are arranged on a straight line inclined at a predetermined angle with respect to the longitudinal direction of the diaper. In this way, the plurality of apertures or cavities is efficiently dispersed in the whole sheet member, and thus it is possible to uniformize strength of the sheet member in a width direction of the diaper (stretching direction of waist stretchable members). In addition, the plurality of apertures or cavities is formed around a joining portion of the sheet member, and thus it is possible to ensure air permeability around this joining portion. Cavities may be provided instead of the apertures in the sheet. Hole bottoms of the cavities are significantly thin. Thus, as with the apertures, it is possible to expect improvement in air permeability of the sheet member. In addition, the inventors have conceived that the problem of the conventional art may be solved based on the above-described information, and completed the invention. Specifically, the invention has a configuration below.

One aspect of the invention relates to a disposable diaper.

The disposable diaper of the invention is divided into a front body part 1, a rear body part 2, and a crotch portion 3 positioned between the front body part 1 and the rear body part 2 in a longitudinal direction (Y direction) thereof. In addition, in the disposable diaper of the invention, a cushion portion 40 is formed in both or any one of the front body part 1 and the rear body part 2.

The cushion portion 40 includes an internal sheet 41, an external sheet 42, one or a plurality of waist stretchable members 43, and a cushion forming sheet 44.

The external sheet 42 is positioned on a non-skin facing surface of the internal sheet 41.

The waist stretchable members 43 are fixed by being interposed between the internal sheet 41 and the external sheet 42 while extending in a width direction (X direction) of the disposable diaper.

The cushion forming sheet 44 overlaps both or any one of the internal sheet 41 and the external sheet 42, and is intermittently joined to these sheets 41 and 42 such that joining portions 51 and non-joining portions 52 are alternately continued in the width direction.

In addition, the cushion forming sheet 44 includes a sheet member in which a plurality of apertures 60 or cavities are regularly formed.

The plurality of apertures 60 or cavities are grouped into apertures 60 or cavities belonging to a row group (R) and apertures 60 or cavities belonging to a column group (C). The row group (R) is a group formed by connecting apertures 60 adjacent to each other in the width direction (X direction). In addition, the column group (C) is a group formed by apertures 60 adjacent to each other in the longitudinal direction (Y direction).

In addition, in the invention, a connection straight line (A) connecting a plurality of apertures 60 or cavities within the column group (C) is inclined at a predetermined angle (θ) with respect to a reference straight line (F) extending in the longitudinal direction (see FIGS. 6(a) and 6(b)).

As in the above-described configuration, in the invention, a pattern of the apertures 60 or cavities formed in the cushion forming sheet 44 is a pattern in which a column of the apertures 60 or cavities is inclined at the predetermined angle (θ) with respect to the virtual reference straight line (F) in the longitudinal direction of the disposable diaper. For this reason, irrespective of a position on the cushion forming sheet 44 at which the reference straight line (F) is drawn, at least one aperture 60 or cavity may be positioned on the reference straight line (F). In addition, according to the above-described pattern, the plurality of apertures 60 or cavities are efficiently dispersed in the width direction (X direction) of the cushion forming sheet 44, and thus strength of the whole cushion forming sheet 44 may be uniformized. Therefore, when the “flute structure” is formed using the cushion forming sheet 44, an internal space inside a ventilation passage easily inflates, and a cushioning property may be enhanced. Further, according to the above-described pattern, the apertures 60 or cavities may be formed around the joining portions 51 in addition to the non-joining portions 52 of the sheets. In particular, the apertures 60 or cavities may overlap or intersect the joining portions 51. In this way, air permeability around the joining portions 51 may be ensured. As a result, air permeability of the whole cushion forming sheet 44 may be improved.

In the disposable diaper of the invention, the predetermined angle (θ) is preferably 5 degrees or more and 45 degrees or less.

When an inclination (θ) of a column of apertures 60 is less than 5 degrees or greater than 45 degrees, the plurality of apertures 60 is unlikely to be dispersed in the whole cushion forming sheet 44. As a result, there is a concern that the ventilation passage of the cushion forming sheet 44 may be unlikely to inflate, or air permeability may be degraded. Therefore, the inclination (θ) of the column of the apertures 60 is preferable 5 degrees or more and 45 degrees or less.

In the disposable diaper of the invention, it is presumed that the virtual reference straight line (F) extending in parallel to the longitudinal direction of the disposable diaper is drawn to simultaneously pass through centers of two or more apertures (61, 62). In this instance, two adjacent apertures on the virtual reference straight line (F) are defined as a first aperture 61 and a second aperture 62, respectively. In this case, an interval (P) between the first aperture 61 and the second aperture 62 in the longitudinal direction is preferably three times or more an interval (D) in the longitudinal direction between adjacent apertures 60 included in the column group (C) (see FIGS. 6(a) and 6(b)).

As in the above-described configuration, the interval (P) in the longitudinal direction between the first aperture 61 and the second aperture 62 positioned on the virtual reference straight line (F) is preferably as long as possible. When the interval (P) is short, a lot of apertures 60 are arranged on one reference straight line (F). However, it is preferable that apertures 60 not be arranged as possible on the one reference straight line (F) to disperse the plurality of apertures 60 in the whole sheet. In this regard, the interval (P) is preferably as long as possible to disperse the plurality of apertures 60.

In the invention, a length (L₁) of the cushion forming sheet (44) in the width direction between the joining portions (51) is preferably longer than a length (L₂) of the internal sheet (41) or the external sheet (42) to which the cushion forming sheet (44) is joined in the width direction between the joining portions (51) (see FIG. 8).

As in the above-described configuration, in the invention, the length of the cushion forming sheet 44 between the intermittent joining portions 51 is preferably longer than the lengths of the sheet members 41 and 42 intermittently joined to the cushion forming sheet 44 between the intermittent joining portions 51 (see FIG. 8). In this way, even when the waist stretchable members 43 do not contract, and the internal sheet 41 and the external sheet 42 are in a stretched state, the cushion forming sheet 44 is in a slightly loose state. For this reason, for example, even when a wearer has a large abdominal circumference, and the waist stretchable members 43 insufficiently contract, the cushion forming sheet 44 forms a wrinkle around a waist of the disposable diaper. Thus, it is possible to exhibit flexibility and a cushioning property. In addition, when the waist stretchable members 43 contract, and a wrinkle is formed in the internal sheet 41 and the external sheet 42 interposing the waist stretchable members 43, a large wrinkle is further formed in the cushion forming sheet 44. In this way, the cushion forming sheet 44 forms a large wrinkle, and thus it is possible to further enhance flexibility and the cushioning property around the waist of the disposable diaper.

A second aspect of the invention relates to a cushion sheet.

The cushion sheet is a sheet-shaped member in which one or a plurality of elastic stretchable members is fixed between a plurality of sheet members. When the plurality of elastic stretchable members is present, the respective elastic stretchable members are disposed in parallel to each other.

The cushion sheet includes an internal sheet 41, an external sheet 42, one or a plurality of elastic stretchable members 43, and a cushion forming sheet 44.

The one or the plurality of elastic stretchable members 43 are fixed by being interposed between the internal sheet 41 and the external sheet 42 while extending in a certain direction (X direction).

The cushion forming sheet 44 overlaps both or any one of the internal sheet 41 and the external sheet 42, and is intermittently joined thereto such that joining portions 51 and non-joining portions 52 are alternately continued in the certain direction (X direction).

In addition, the cushion forming sheet 44 includes a sheet member in which a plurality of apertures 60 or cavities are regularly formed.

The plurality of apertures 60 or cavities are grouped into a row group (R) and a column group (C).

The row group (R) is a group formed by connecting apertures 60 or cavities adjacent to each other in the certain direction (X direction).

The column group (C) is a group formed by connecting apertures 60 or cavities adjacent to each other in a perpendicular direction (Y direction) perpendicular to the certain direction.

Herein, a connection straight line (A) connecting a plurality of apertures 60 or cavities within the column group (C) is inclined at a predetermined angle (θ) with respect to a reference straight line (F) extending in the perpendicular direction (Y direction).

As in the above-described configuration, a cushion sheet having a high cushioning property may be manufactured as a separate body from a disposable diaper.

### Advantageous Effects of Invention

According to the invention, it is possible to uniformize strength of an aperture sheet employed in a so-called “flute structure”, and improve air permeability around a joining portion.

### BRIEF DESCRIPTION OF DRAWINGS

FIG. 1 is an external perspective view of a disposable diaper according to an embodiment of the invention.

FIG. 2 is a developed plan view of the disposable diaper according to the embodiment of the invention.

FIG. 3 is a cross-sectional view taken along Y-Y line illustrated in FIG. 2.

FIGS. 4(a) to 4(d) conceptually illustrate a structure of a cushion portion. FIG. 4(a) schematically illustrates a component of the cushion portion, and FIGS. 4(b) to 4(d) schematically illustrates cross section states of the cushion portion.

FIG. 5 is a perspective view schematically illustrating the cushion portion.

FIGS. 6(a) and 6(b) illustrate an example of an aperture pattern of a cushion forming sheet.

FIGS. 7(a) to 7(d) conceptually illustrate a structure of a cushion portion according to another embodiment. FIG. 7(a) schematically illustrates a component of the cushion portion, and FIGS. 7(b) to 7(d) schematically illustrates cross section states of the cushion portion.

FIG. 8 is an explanatory drawing for description of a concept of a length (L₁) and a length (L₂).

FIGS. 9(a) and 9(b) illustrate other embodiments of the cushion portion.

FIGS. 10(a) and 10(b) illustrate other embodiments of the cushion portion.

FIGS. 11(a) and 11(b) illustrate other embodiments of the cushion portion.

### DESCRIPTION OF EMBODIMENTS

Hereinafter, embodiments for implementing the invention will be described using drawings. The invention is not restricted to a mode described below, and includes a mode appropriately corrected from the mode below within a clear range by those skilled in the art.

In this specification, a “front body part” refers to a part that covers a belly side (a front of a body) of a wearer when the wearer wears a diaper. In addition, a “crotch portion” refers to a portion that covers a crotch of the wearer when the wearer wears the diaper. In addition, a “rear body part” refers to a part that covers a back side (a rear of the body) of the wearer when the wearer wears the diaper.

In addition, in this specification, a “longitudinal direction” basically refers to a direction (Y direction) of connecting a front body part to a rear body part in a disposable diaper. In addition, a “width direction” basically refers to a direction (X direction) perpendicular to the longitudinal direction of the disposable diaper.

In addition, in this specification, “A to B” indicates “A or more and B or less”.

### (1. Overall Configuration of Disposable Diaper)

For example, the invention is widely applicable to a known pants-type disposable diaper and a known tape-type disposable diaper. The pants-type disposable diaper is a disposable diaper in which both side portions corresponding to a front body part and a rear body part are joined to each other in advance (for example, Patent Literature 1). In addition, the tape-type disposable diaper is a disposable diaper in which an adhesive tape is attached to both side portions corresponding to a front body part or a rear body part, and the front body part and the rear body part are attached to each other using an adhesive tape at the time of wearing (for example, Patent Literature 3). In addition, examples of the pants-type disposable diaper include an integrated disposable diaper (Patent Literature 1) in which an outer packaging body is one body across the rear body part from the front body part, and a separation-type disposable diaper (Patent Literature 2) in which an outer packaging body of the front body part and an outer packaging body of the rear body part are separated from each other. Hereinafter, content of the invention will be specifically described using, as an example, the separation-type disposable diaper of the pants-type disposable diaper among these various types of disposable diapers.

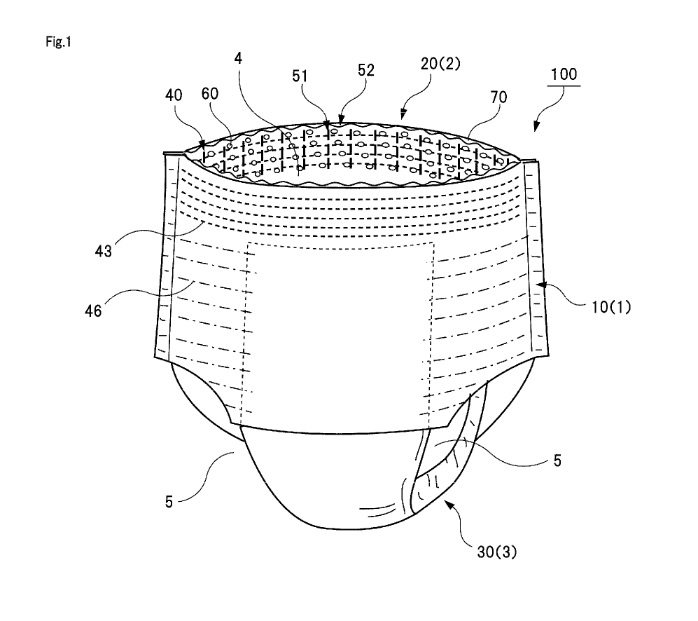

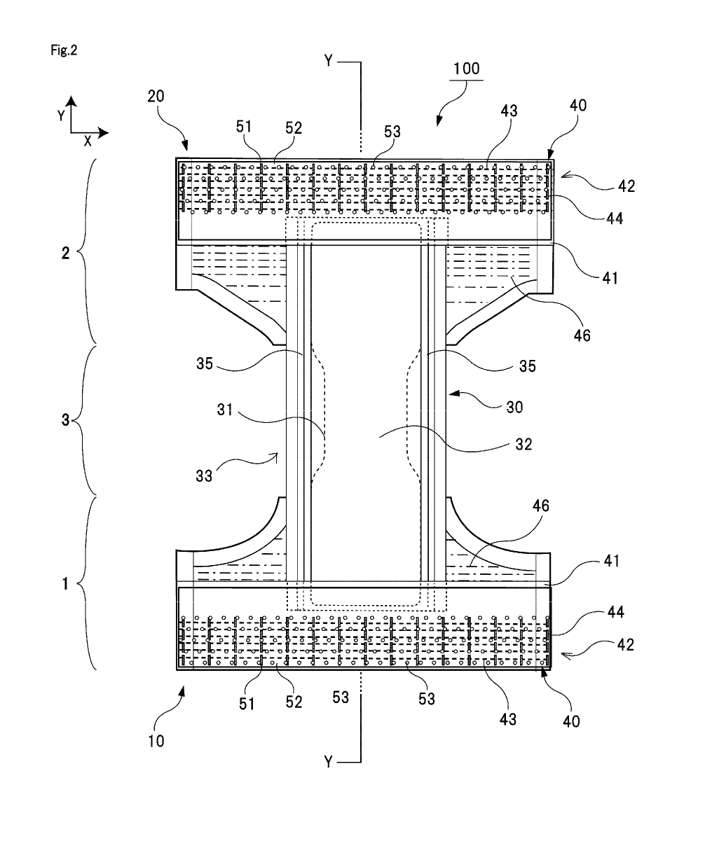

FIG. 1 is a perspective view illustrating an example of a disposable diaper 100 viewed from a front body part side. In addition, FIG. 2 is a developed view illustrating an example of the disposable diaper, and illustrates a state in which the disposable diaper is viewed from a skin facing surface side. As illustrated in the developed view of FIG. 2, the disposable diaper 100 is divided into a front body part 1 coming into contact with a belly portion of a wearer at the time of wearing, a rear body part 2 coming into a back portion of the wearer, and a crotch portion 3 fit to a crotch of the wearer in the longitudinal direction. In the present embodiment, the disposable diaper 100 has the front body part 1 that includes a front body part outer packaging body 10 and the rear body part 2 that includes a rear body part outer packaging body 20. Then, an absorbent main body 30 is bridged in the crotch portion 3 between the front body part outer packaging body 10 and the rear body part outer packaging body 20. That is, one end of the absorbent main body 30 is fixed to the front body part outer packaging body 10, and the other end of the absorbent main body 30 is fixed to the rear body part outer packaging body 20.

The diaper is assembled from a developed state illustrated in FIG. 2 to a state illustrated in FIG. 1 when both end portions of the front body part outer packaging body 10 and the rear body part outer packaging body 20 in the width direction are joined to each other. As illustrated in FIG. 1, a waist opening 4 is formed by an upper edge of the front body part outer packaging body 10 and an upper edge of the rear body part outer packaging body 20. In addition, when the both end portions of the front body part outer packaging body 10 and the rear body part outer packaging body 20 are joined to each other, a leg opening 5 positioned around a leg portion of the wearer at the time of wearing is formed. For this reason, the wearer may wear the disposable diaper 100 by inserting both leg portions from the waist opening 4 and withdrawing each leg portion from the leg opening 5.

In addition, as illustrated in FIG. 2, in the present embodiment, a cushion portion 40 is formed in each of the front body part outer packaging body 10 and the rear body part outer packaging body 20. The cushion portion 40 is formed to extend in the width direction along an end edge that forms the waist opening 4. The cushion portion 40 includes a plurality of waist stretchable members 43. The plurality of waist stretchable members 43 extend in the width direction of the disposable diaper along an end portion of the front body part outer packaging body 10 and an end portion of the rear body part outer packaging body 20 corresponding to an opening edge of the waist opening 4. The plurality of waist stretchable members 43 is fixed in an extended state, and a waist gather is formed around the waist opening 4 when the plurality of waist stretchable members 43 contracts.

In the present embodiment, the cushion portion 40 is provided in each of the front body part outer packaging body 10 and the rear body part outer packaging body 20. However, in the invention, the cushion portion 40 may be provided in at least one of the front body part outer packaging body 10 and the rear body part outer packaging body 20.

### (2. Configuration of Each Portion of Disposable Diaper)

Next, a description will be given of an example of a configuration of each portion of the disposable diaper 100 using drawings.

### (2-1. Cushion Portion)

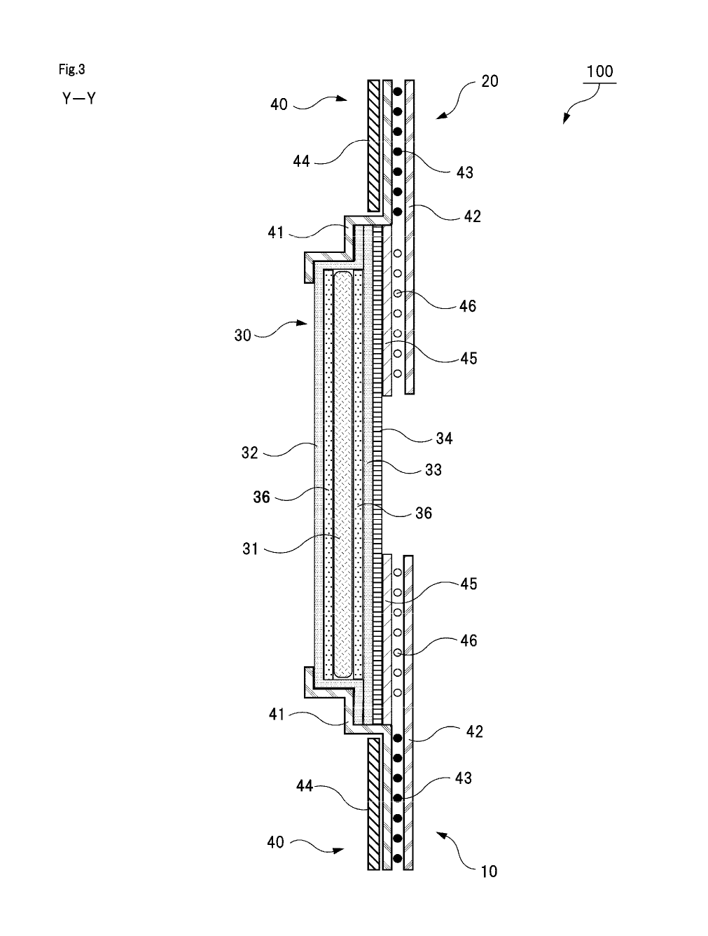

FIG. 3 illustrates a cross-sectional view taken along Y-Y line illustrated in FIG. 2. Even though the disposal diaper is drawn by assigning conceptual thicknesses to various sheet members included in the disposable diaper in the cross-sectional view of FIG. 3, the sheet members included in the disposable diaper are normally significantly thin.

As illustrated in FIG. 3, the front body part outer packaging body 10 and the rear body part outer packaging body 20 (hereinafter collectively referred to as outer packaging bodies 10 and 20) include the cushion portion 40. The cushion portion 40 is a portion that forms a waist gather at a rim of the waist opening 4 of the disposable diaper, and includes a plurality of sheet members and stretchable members.

As illustrated in the cross-sectional view of FIG. 3, the cushion portion 40 includes an internal sheet 41 positioned on a skin facing surface side, an external sheet 42 positioned on a non-skin facing surface side, and a plurality of waist stretchable members 43 fixed between the internal sheet 41 and the external sheet 42. As illustrated in FIG. 3, the internal sheet 41 and the external sheet 42 together form an end edge of the disposable diaper in the longitudinal direction. That is, the internal sheet 41 and the external sheet 42 form an end edge along the waist opening 4 of the disposable diaper. In addition, as illustrated in a plan view of FIG. 2, the plurality of waist stretchable members 43 extend in parallel in the width direction of the disposable diaper. In addition, the plurality of waist stretchable members 43 is disposed with predetermined intervals in the longitudinal direction of the disposable diaper. In the example illustrated in FIG. 3, the internal sheet 41 and the external sheet 42 are separated from each other. However, the sheets 41 and 42 may be connected and integrally formed. In more detail, one sheet member may be folded back at a waist end edge, thereby setting a portion on the skin facing surface side to the internal sheet 41, and setting a portion on the non-skin facing surface side to the external sheet 42.

The plurality of waist stretchable members 43 is fixed between the internal sheet 41 and the external sheet 42 while being stretched in the width direction. For example, when the plurality of waist stretchable members 43 is fixed, an elongation percentage is preferable in a range of 150 to 400%, 200 to 380%, or 250 to 360%. The “elongation percentage” refers to a value indicating a length of the stretchable member in an extended state when a length (full size) of the stretchable member in a non-extended state (a free state) is set to 100%. Since the waist stretchable member 43 in the extended state is fixed between the internal sheet 41 and the external sheet 42 in this way, a wrinkle (gather) is formed in the internal sheet 41 and the external sheet 42 when the waist stretchable members 43 contract.

When the waist stretchable members 43 is fixed by being interposed between the internal sheet 41 and the external sheet 42, for example, an adhesive may be applied to the waist stretchable members 43, and the waist stretchable members 43 to which the adhesive is applied may be interposed between the internal sheet 41 and the external sheet 42. In addition, for example, after the adhesive is applied to both or one of the internal sheet 41 and the external sheet 42, the waist stretchable members 43 may be fixed by being interposed between the sheets 41.

Further, in the present embodiment, the cushion portion 40 includes a cushion forming sheet 44 disposed on the skin facing surface side of the internal sheet 41. The cushion forming sheet 44 is a sheet member directly coming into contact with a skin of the wearer of the disposable diaper, and has a function of forming a wrinkle (gather) on the skin facing surface side of the internal sheet 41. The cushion forming sheet 44 may be further disposed on the non-skin facing surface side of the external sheet 42. In addition, the cushion forming sheet 44 overlapping the internal sheet 41 and the cushion forming sheet 44 overlapping the external sheet 42 (not illustrated) may be integrally connected to each other.

In addition, as illustrated in FIG. 1 and FIG. 2, the cushion forming sheet 44 includes a sheet member in which a plurality of apertures 60 is formed. In addition, although not illustrated, cavities may be formed instead of the apertures 60 in the cushion forming sheet 44. Herein, the apertures refer to through-holes formed to fully penetrate the sheet member. Meanwhile, even though the cavities are not through-holes, the cavities refer to portions in which a thickness of the sheet member is thinner than that in another portion. Air permeability is enhanced when the apertures 60 are formed in the cushion forming sheet 44. However, improvement in air permeability is expected when the cavities are formed in the cushion forming sheet 44. Hereinafter, a detailed description will be given of an example of the cushion forming sheet 44 in which the plurality of apertures 60 is formed. However, in this specification, the cavities may be used as a replacement in a description of the apertures 60 formed in the cushion forming sheet 44. In addition, both the apertures 60 and the cavities may be formed in the cushion forming sheet 44.

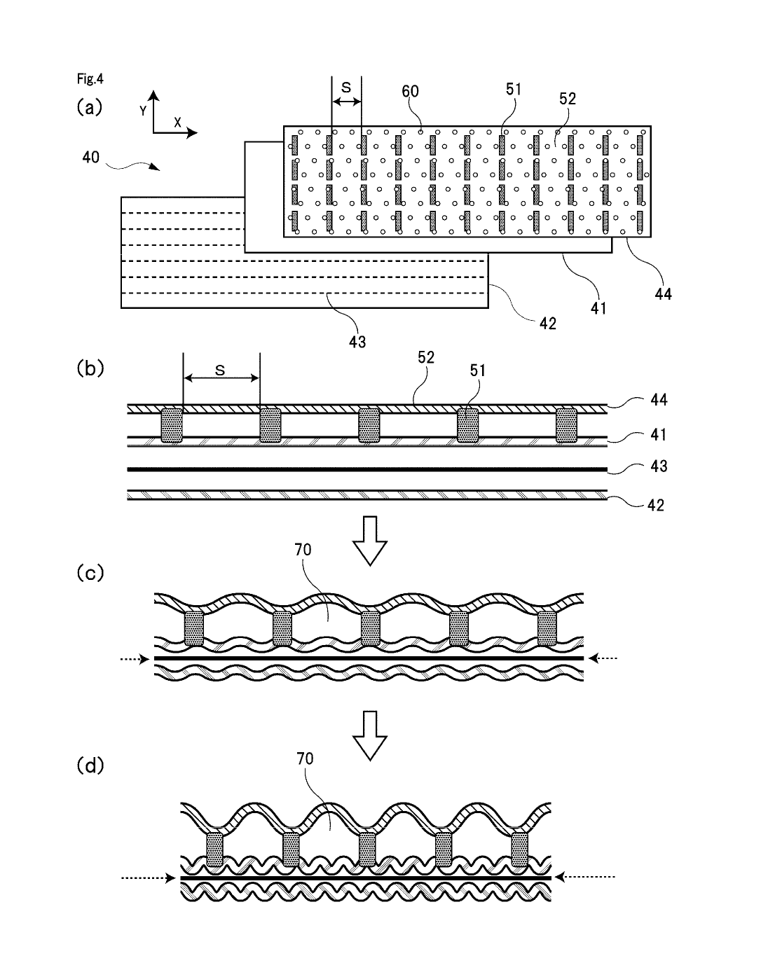

Herein, a description will be given of a structure in which the cushion forming sheet 44 included in the cushion portion 40 forms a wrinkle with reference to FIGS. 4(a) to 4(d). FIGS. 4(a) to 4(d) are schematic views conceptually illustrating various components included in the cushion portion 40. As illustrated in FIG. 4(a), the cushion portion 40 includes the internal sheet 41, the external sheet 42, the plurality of waist stretchable members 43 fixed between these sheets 41 and 42, and the cushion forming sheet 44 overlapping the internal sheet 41. Further, the plurality of apertures 60 is formed in the cushion forming sheet 44. The cushion forming sheet 44 may overlap the external sheet 42, or overlap both the internal sheet 41 and the external sheet 42.

As illustrated in FIG. 4(a), joining portions 51 of the cushion forming sheet 44 and the internal sheet 41 are intermittently formed in the width direction (X direction) of the diaper. That is, the cushion forming sheet 44 and the internal sheet 41 are joined to each other by alternately forming the joining portions 51 and non-joining portions 52 in the width direction of the diaper. In the joining portions 51, the cushion forming sheet 44 and the internal sheet 41 may be joined to each other using a hot-melt adhesive, etc., or the cushion forming sheet 44 and the internal sheet 41 may be welded by heat sealing or ultrasonic sealing. As illustrated in FIG. 4(a), basically, the joining portions 51 are linearly formed in the longitudinal direction (Y direction) of the diaper. That is, each of the joining portions 51 extends in parallel to the longitudinal direction (Y direction) of the diaper. The joining portions 51 may have a linear shape, a dotted shape (dot shape), or a rectangular shape. In addition, as illustrated in FIGS. 4(a) to 4(d), the joining portions 51 may be intermittently formed in the longitudinal direction (Y direction) of the diaper. For example, when the joining portions 51 have the linear shape, the linear joining portions 51 extend in parallel to the longitudinal direction (Y direction) of the disposable diaper. Alternatively, when the joining portions 51 have the dotted shape or the rectangular shape, a plurality of joining portions 51 forms a column, and the column of the joining portions 51 extends in parallel to the longitudinal direction (Y direction) of the disposable diaper. Besides, a known joining mode may be appropriately employed as a joining mode for the cushion forming sheet 44 and the internal sheet 41 with reference to Patent Literature 4 (JP 2011-254996 A).

FIG. 4(b) illustrates a cross-sectional view in a state in which the external sheet 42, the waist stretchable members 43, the internal sheet 41, and the cushion forming sheet 44 overlap one another in order from a lower side (non-skin contact surface side) of the figure. FIG. 4(b) illustrates a state in which the waist stretchable members 43 do not contract. As illustrated in FIG. 4(b), the waist stretchable members 43 are fixed by being interposed between the internal sheet 41 and the external sheet 42 in the extended state. In addition, the cushion forming sheet 44 is joined to a skin facing surface side of the internal sheet 41 in the joining portions 51 intermittently formed in the width direction.

Next, FIG. 4(c) illustrates an appearance in which the waist stretchable members 43 slightly contract, and FIG. 4(b) illustrates an appearance in which the waist stretchable members 43 greatly contract. As illustrated in FIGS. 4(c) and 4(d), in response to contraction of the waist stretchable members 43, a wrinkle is formed in the cushion forming sheet 44. That is, when the waist stretchable members 43 contract, the cushion forming sheet 44 is separated from the internal sheet 41 in the non-joining portion 52, and thus a ventilation passage (space) 70 is formed between the cushion forming sheet 44 and the internal sheet 41. Such a structure is referred to as a “flute structure” in this specification. When the ventilation passage 70 is formed between the intermittently formed joining portions 51 in this way, hardness of the internal sheet 41 interposing the waist stretchable members 43 is unlikely to be directly delivered to the skin of the wearer. In addition, when the “flute structure” is formed, air permeability, flexibility, and a cushioning property may be assigned to the whole waist gather.

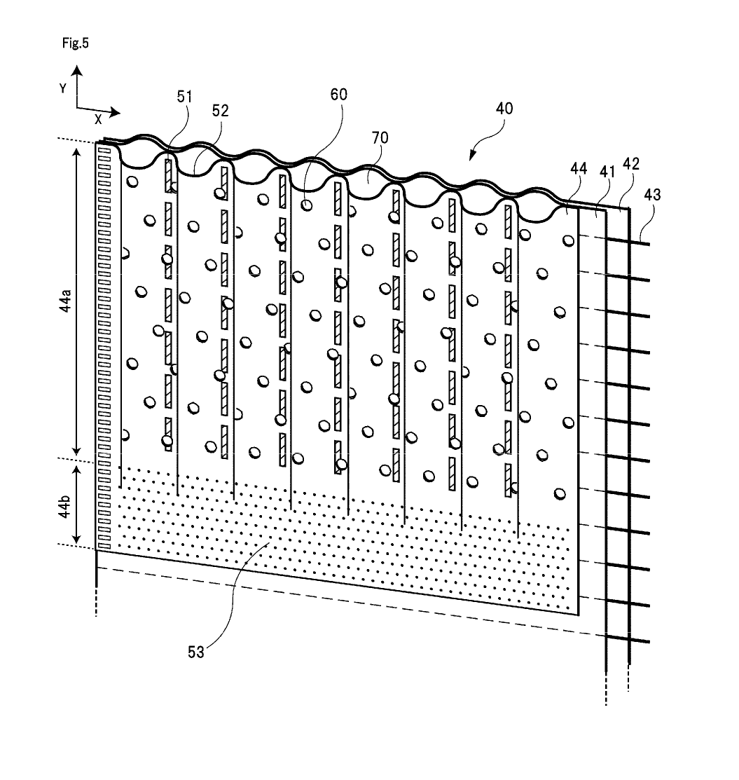

FIG. 5 is a perspective view schematically illustrating a structure of the cushion portion 40. As illustrated in FIG. 5, when the waist stretchable members 43 contract, the cushion forming sheet 44 is separated from the internal sheet 41 in a non-joining portion 52 between adjacent joining portions 51. For this reason, the ventilation passage (space) 70 is formed between the cushion forming sheet 44 and the internal sheet 41. In addition, the plurality of apertures 60 is formed in the cushion forming sheet 44. The apertures 60 penetrate the cushion forming sheet 44, and are connected to an inside of the ventilation passage 70. For this reason, air is introduced to the inside of the ventilation passage 70 or air inside the ventilation passage 70 is discharged to the outside through the apertures 60. Therefore, when the plurality of apertures 60 is formed in the cushion forming sheet 44, air permeability of the cushion forming sheet 44 is improved. In addition, when the plurality of apertures 60 is formed, a touch of the cushion forming sheet 44 may be further softened.

In addition, as illustrated in FIG. 5, the cushion forming sheet 44 is vertically divided into an intermittent joining region 44a and a full joining region 44b. The intermittent joining region 44a is provided at a position (upper portion of FIG. 5) close to the waist end edge of the disposable diaper, and the full joining region 44b is provided at a position (lower portion of FIG. 5) close to the crotch portion 3 of the disposable diaper. As illustrated in FIG. 5, in the intermittent joining region 44a, as described in the foregoing, the cushion forming sheet 44 is joined to the internal sheet 41 by the plurality of joining portions 51 intermittently provided in the width direction. For this reason, the above-described “flute structure” is formed in the intermittent joining region 44a when the waist stretchable members 43 contract. Meanwhile, the cushion forming sheet 44 is fully joined to the internal sheet 41 by an adhesive 53, etc. in the full joining region 44b. That is, even when the waist stretchable members 43 contract, the cushion forming sheet 44 is not separated from the internal sheet 41 in the full joining region 44b. For this reason, the ventilation passage (space) 70 is not formed between the cushion forming sheet 44 and the internal sheet 41 in the full joining region 44b.

When the full joining region 44b is provided in the lower portion of the cushion forming sheet 44 in this way, the cushion forming sheet 44 is firmly fixed to the internal sheet 41 and is unlikely to peel off. In more detail, a lower end side of the cushion forming sheet 44 is frequently rubbed with the skin of the wearer of the diaper, and thus is a region which relatively easily peels off. For this reason, when the intermittent joining portions 51 are formed up to the lower end side of the cushion forming sheet 44, there is a concern that the cushion forming sheet 44 may peel off, thereby giving the wearer an unpleasant feeling or causing incontinence. In this regard, to prevent such a case, it is preferable to fully apply the adhesive 53, etc. to a region of the lower portion of the cushion forming sheet 44 to firmly fix the cushion forming sheet 44 to the internal sheet 41. However, when the full joining region 44b is provided in the lower portion of the cushion forming sheet 44, a lower portion of the ventilation passage 70 of the “flute structure” is blocked, and thus there is a concern that air may be unlikely to enter and leave the inside of the ventilation passage 70. In this regard, in the invention, the plurality of apertures 60 is formed in the cushion forming sheet 44 in order to maintain air permeability by allowing air to enter and leave the inside of the ventilation passage 70 even when the lower portion of the ventilation passage 70 is blocked in this way. Air permeability of the cushion forming sheet 44 may be maintained by forming the plurality of apertures 60 in the cushion forming sheet 44 even when the full joining region 44b is provided in the lower portion of the cushion forming sheet 44.

Besides, it is preferable to apply filiform elastic gum or belt-like elastic gum as the waist stretchable members 43. A material such as styrene rubber, olefin rubber, urethane rubber, ester rubber, polyurethane, polyethylene, polystyrene, styrene-butadiene, silicone, polyester, etc. may be used as such a rubber material.

In addition, in the present embodiment, as illustrated in FIG. 3, the internal sheet 41 extends toward the crotch portion 3 of the diaper. Further, a portion of the internal sheet 41 overlaps an upper part (skin facing surface) of an end portion of the absorbent main body 30 in the longitudinal direction. When the portion of the internal sheet 41 is overlapped with the absorbent main body 30, the absorbent main body 30 is firmly fixed to the outer packaging bodies 10 and 20 and is unlikely to be removed.

In addition, the outer packaging bodies 10 and 20 may further include an inner sheet 45 and a stretchable tammy member 46. In more detail, as illustrated in FIG. 3, in the present embodiment, the external sheet 42 extends toward the crotch portion 3, and a portion thereof is positioned on the non-skin facing surface of the absorbent main body 30. Herein, the inner sheet 45 is disposed on the skin facing surface side of the extending portion of the external sheet 42. Further, one or a plurality of stretchable tammy members 46 is fixed by being interposed between the extending portion of the external sheet 42 and the inner sheet 45. As with the waist stretchable members 43, the stretchable tammy member 46 is fixed between the extending portion of the external sheet 42 and the inner sheet 45 in an extended state in the width direction of the diaper.

### (2-1-1. Aperture Pattern of Cushion Forming Sheet)

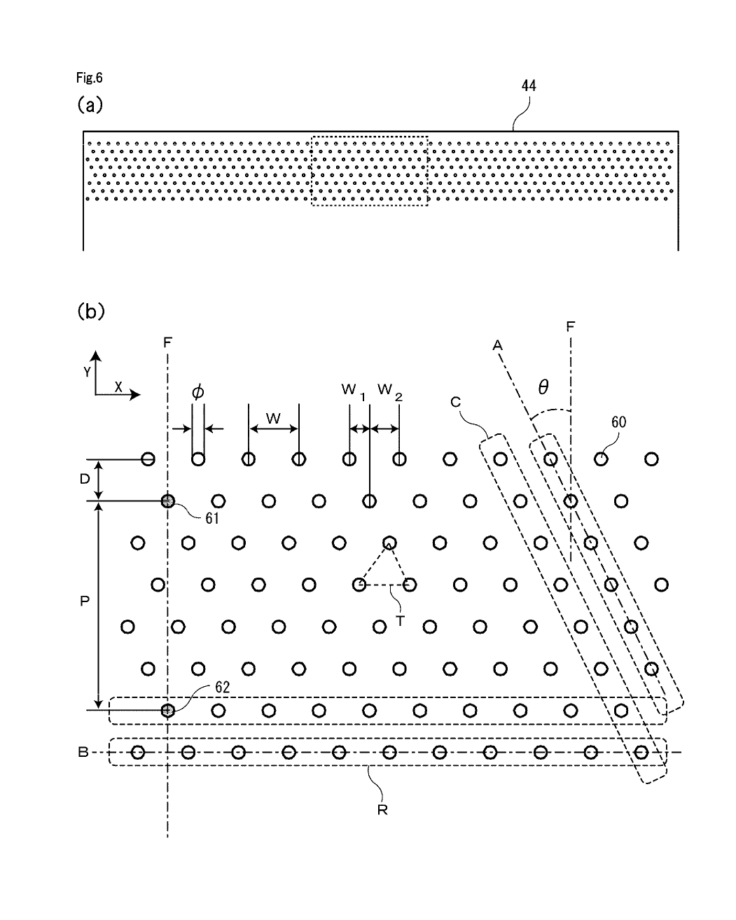

Next, a description will be given of a preferable pattern of the apertures 60 of the cushion forming sheet 44 with reference to FIGS. 6(a) and 6(b). FIG. 6(a) illustrates an example of a pattern of the apertures 60, and FIG. 6(b) illustrates an enlarged view of an inside a frame indicated by a dotted line illustrated in FIG. 6(a). In the example illustrated in FIGS. 6(a) and 6(b), shapes of the apertures 60 correspond to a perfect circle. However, the shapes of the apertures 60 may correspond to an oval or a polygon such as a triangle, a rectangle, a pentagon, a hexagon, etc.

As illustrated in FIG. 6(b), the plurality of apertures 60 is formed in a regular pattern in the cushion forming sheet 44. First, the plurality of apertures 60 may be grouped into a row group (R) and a column group (C). The row group (R) refers to a group formed by connecting apertures 60 adjacent in the width direction (X direction) of the diaper. That is, the row group (R) refers to a group of apertures 60 formed when apertures 60 closest to each other are connected to each other from side to side in the width direction (X direction). Meanwhile, the column group (C) refers to a group formed by connecting apertures 60 adjacent in the longitudinal direction (Y direction) of the diaper. That is, the column group (C) refers to a group of apertures 60 formed when apertures 60 closest to each other are connected to each other up and down in the longitudinal direction (Y direction). A plurality of row groups (R) and a plurality of column groups (C) are present. In addition, each of the row group (R) and the column group (C) has a linear shape. In addition, one aperture 60 belongs to one of the plurality of row groups (R) and one of the plurality of column groups (C).

In the example illustrated in FIG. 6(b), all the plurality of row groups (R) extend in parallel to each other in the width direction (X direction) of the disposable diaper. That is, when a plurality of apertures 60 belonging to one row group (R) is connected by a virtual connection line (B), this connection line (B) extends in parallel in the width direction (X direction). In this way, all the rows (R) of the apertures 60 are preferably parallel to the width direction (X direction) of the diaper.

Meanwhile, in the present invention, all the plurality of column groups (C) are inclined at a predetermined angle (θ) with respect to the longitudinal direction (Y direction) of the disposable diaper. Specifically, a virtual connection line (A) is drawn to connect a plurality of apertures 60 belonging to one column group (C). In addition, a virtual reference straight line (F) is drawn in parallel to the longitudinal direction (Y direction) of the disposable diaper. In this instance, an angle (acute angle) formed by the connection line (A) and the reference straight line (F) corresponds to the inclined angle (θ) of the column group (C) of the apertures 60. In this way, a characteristic of the invention is that the column (C) of the apertures 60 is inclined at the predetermined angle (θ).

For example, the inclined angle (θ) of the column group (C) is preferably in a range of 5 to 45 degrees. In addition, the inclined angle (θ) is preferably less than 45 degrees, that is, not 45 degrees. For example, the inclined angle (θ) is particularly preferably in a range of 10 to 43 degrees, 15 to 40 degrees, or 20 to 35 degrees.

When the column (C) of the apertures 60 is inclined in this way, it is possible to eliminate a region in which no aperture 60 is present in the longitudinal direction (Y direction) of the disposable diaper. In more detail, as illustrated in FIGS. 6(a) and 6(b), when the virtual reference straight line (F) is drawn in parallel to the longitudinal direction (Y direction) of the disposable diaper, a portion (which may not be a center) of at least one aperture 60 is positioned on the reference straight line (F). In this way, the cushion forming sheet 44 in which such an aperture pattern is formed has nearly uniform strength in the width direction (X direction) of the diaper. As a result, when the waist stretchable members 43 contract in the width direction (X direction) of the diaper, a contractile force thereof is uniformly delivered to the whole cushion forming sheet 44, and the space formed by the cushion forming sheet 44 efficiently inflates. In addition, air permeability of the cushion forming sheet 44 may be improved by allowing a portion of at least one aperture 60 to be positioned on a certain reference straight line (F) as in the pattern illustrated in FIGS. 6(a) and 6(b).

A preferable pattern of the aperture 60 will be descried in more detail.

As illustrated in FIGS. 6(a) and 6(b), all intervals (W) between adjacent apertures 60 in the width direction (Y direction) of the diaper are equal to each other. For example, an interval (W) of the apertures 60 in the width direction is preferably smaller than an interval (S) of the joining portions 51 in the width direction (see FIGS. 4(a) to 4(d)) (W<S). For example, when the interval (S) of the joining portions 51 is set to 100%, the interval (W) of the apertures 60 may be in a range of 10 to 95% or 20 to 90%. In particular, the interval (W) is preferably in a range of 50 to 85% or 60 to 80%. Specific numerical values are shown below. The interval (S) of the joining portions 51 in the width direction (see FIGS. 4(a) to 4(d)) is preferably in a range of 5 to 20 mm, 6 to 18 mm, or 7 to 16 mm. In addition, the interval (W) of the apertures 60 in the width direction is preferably in a range of 3 to 16 mm or 4 to 14 mm. When the interval (W) of the apertures 60 is set in the range, and when the ventilation passage 70 is formed by the cushion forming sheet 44, at least one aperture 60 is present on the ventilation passage 70.

In addition, as illustrated in FIGS. 6(a) and 6(b), an interval between an aperture 60 belonging to a certain row group (R) and an aperture 60 belonging to a row group (R) adjacent to the certain row group (R) is indicated by symbols W₁ and W₂. A sum of the interval (W₁) and the interval (W₂) is equal to the interval (W). In this instance, the interval (W₁) and the interval (W₂) preferably correspond to different values (W₁≠W₂). In FIG. 6, a short interval is indicated by the symbol W₁, and a long interval is indicated by symbol W₂ (W₁)<W₂). The short interval (W₁) is an interval in the width direction between apertures 60 adjacent to each other within the same column group (C). The long interval (W₂) is an interval in the width direction between apertures 60 belonging to different column groups (C) adjacent to each other. Herein, when the interval (W₁) and the interval (W₂) correspond to the same value (W₁=W₂), there is a possibility that a region in which no aperture 60 is present in the longitudinal direction (Y direction) of the disposable diaper may be formed. Such a state needs to be avoided. Therefore, in the invention, it is preferable that the interval (W₁) and the interval (W₂) be intentionally set to different values (W₁≠W₂, that is, W₁<W₂). For example, the short interval (W₁) is preferably in a range of 10 to 95% or 20 to 90%, and is particularly preferably in a range of 50 to 85% or 60 to 80% with respect to the long interval (W₂). When such a condition (W₁<W₂) is satisfied, a triangle (T) formed by connecting three closest apertures 60 by a line does not correspond to a regular triangle and an isosceles triangle. That is, in the invention, the triangle (T) preferably corresponds to a triangle other than the regular triangle and the isosceles triangle since there is a possibility that a region in which no aperture 60 is present may be formed in the longitudinal direction (Y direction) when the triangle (T) corresponds to the regular triangle or the isosceles triangle.

In FIGS. 6(a) and 6(b), an interval in the longitudinal direction (Y direction) between adjacent apertures 60 belonging to the column group (C) is indicated by a symbol D. The interval (D) between the apertures 60 in the longitudinal direction may be arbitrarily set in a range satisfying the above-described conditions (conditions of θ, W, W₁, W₂, etc.). In the pattern illustrated in FIGS. 6(a) and 6(b), all intervals (D) of apertures 60 in the width direction (Y direction) are equal to each other. In addition, for example, the interval (D) between the apertures 60 in the longitudinal direction is preferably in a range of 70 to 130% or 80 to 120% with respect the interval (W) of the apertures 60 in the width direction. When the interval (D) between the apertures 60 in the longitudinal direction is set to be nearly equal to the interval (W) of the apertures 60 in the width direction, the pattern of the apertures 60 becomes a balanced clean pattern as illustrated in FIG. 6(a), and an excellent appearance is obtained.

In addition, in FIGS. 6(a) and 6(b), when one reference straight line (F) is drawn in parallel to the longitudinal direction (Y direction) of the diaper, two adjacent apertures on the reference straight line (F) are indicated by reference numeral 61 and reference numeral 62, respectively. The two adjacent apertures on the reference straight line (F) refer to two apertures having a shortest distance therebetween among a plurality of apertures positioned on the reference straight line (F). In the pattern illustrated in FIGS. 6(a) and 6(b), a center of a first aperture 61 and a center of a second aperture 62 are positioned on one reference straight line (F). In FIGS. 6(a) and 6(b), the first aperture 61 and the second aperture 62 are conveniently indicated in black. In addition, in FIGS. 6(a) and 6(b), an interval in the longitudinal direction (Y direction) between the first aperture 61 and the second aperture 62 is indicated by a symbol P. The interval (P) between the first aperture 61 and the second aperture 62 is longer than the interval (D) between the apertures 60 in the longitudinal direction described above (D<P). For example, the interval (P) is preferable three times or more the interval (D), and particularly preferably four times or more or five times or more the interval (D). In the example illustrated in FIGS. 6(a) and 6(b), the interval (P) is set to be five times the interval (D).

In this way, the interval (P) between the first aperture 61 and the second aperture 62 adjacent to each other on a certain reference straight line (F) is preferably as long as possible. When the interval (P) is short, there is a possibility that a lot of apertures 60 may be arranged on one reference straight line (F). When a lot of (for example, three or more) apertures 60 are arranged on one reference straight line (F), the plurality of apertures 60 is unlikely to be dispersed in the whole sheet. Then, unevenness (deviation) may be generated in strength of the cushion forming sheet 44 in the width direction, or a portion having excellent air permeability and a portion having poor air permeability are clearly distinguished. Therefore, the plurality of apertures 60 is preferably dispersed in the whole sheet as much as possible in order to uniformize strength or air permeability of the cushion forming sheet 44. To this end, the apertures 60 may as well not be arranged along one reference straight line (F). Therefore, the interval (P) is preferably as long as possible to disperse the plurality of apertures 60. A mode in which any center of an aperture 60 is not present on one reference straight line (F) passing through a center of a certain aperture 60 is a preferable mode of the invention.

In addition, in FIGS. 6(a) and 6(b), a diameter of the aperture 60 is indicated by a symbol φ. For example, the diameter (φ) of the aperture 60 is preferably in a range of 5 to 50%, 7 to 40%, or 10 to 30% with respect to the interval (W) of the apertures in the width direction of the aperture 60. Specifically, the diameter (φ) of the aperture 60 is preferably 0.5 mm or more, and particularly preferably in a range of 0.5 to 3.0 mm, 0.8 to 2.0 mm, or 1.0 to 1.5 mm.

In addition, a description will be given of a relation between the pattern of the apertures 60 and a pattern of the joining portions 51. For example, as illustrated in FIG. 4(a) and FIG. 5, a column of the joining portions 51 is disposed to extend in parallel in the longitudinal direction (Y direction) of the diaper. That is, the column of the joining portions 51 is disposed in parallel to the virtual reference straight line (F) illustrated in FIGS. 6(a) and 6(b). In contrast, a column of the apertures 60 is disposed to be inclined with respect to the longitudinal direction (that is, the reference straight line F) of the diaper. For this reason, a direction in which the column of the plurality of apertures 60 extends is inclined at a predetermined angle with respect to a direction in which the column of the joining portions 51 extends. As a result, in the invention, a region in which the apertures 61 overlap or intersect the joining portions 51 is present. In this way, when the apertures 60 are allowed to partially overlap or intersect the joining portions 51, air permeability around the joining portions 51 may be ensured. In this way, air permeability of the whole cushion forming sheet 44 is improved.

When the pattern of the apertures 60 is determined based on the above-described various conditions, the preferable pattern illustrated in FIGS. 6(a) and 6(b) may be formed. The pattern of the apertures 60 illustrated in FIGS. 6(a) and 6(b) may prevent unevenness (deviation) from being generated in strength of the cushion forming sheet 44 in the width direction. For this reason, the cushion forming sheet 44 in which such a pattern is formed may form the ventilation passage 70 having an excellent cushioning property since the space efficiently inflates when the waist stretchable members 43 contract. In addition, the cushion forming sheet 44 in which such a pattern is formed is generally excellent in air permeability. In particular, when the apertures 60 partially overlap or interest the joining portions 51, the cushion forming sheet 44 exhibits preferable air permeability around the joining portions 51.

Besides, each of the internal sheet 41, the external sheet 42, and the cushion forming sheet 44 is preferably formed using a material such as a nonwoven fabric having an excellent touch and air permeability. For example, a nonwoven fabric manufactured using a spunbond method, an air-through method, or an air-laid method may be used. In addition, apertures having the above-described pattern may be formed in the cushion forming sheet 44 using a shedding mechanism of a known punching scheme.

### (2-1-2. Change of Length of Cushion Forming Sheet)

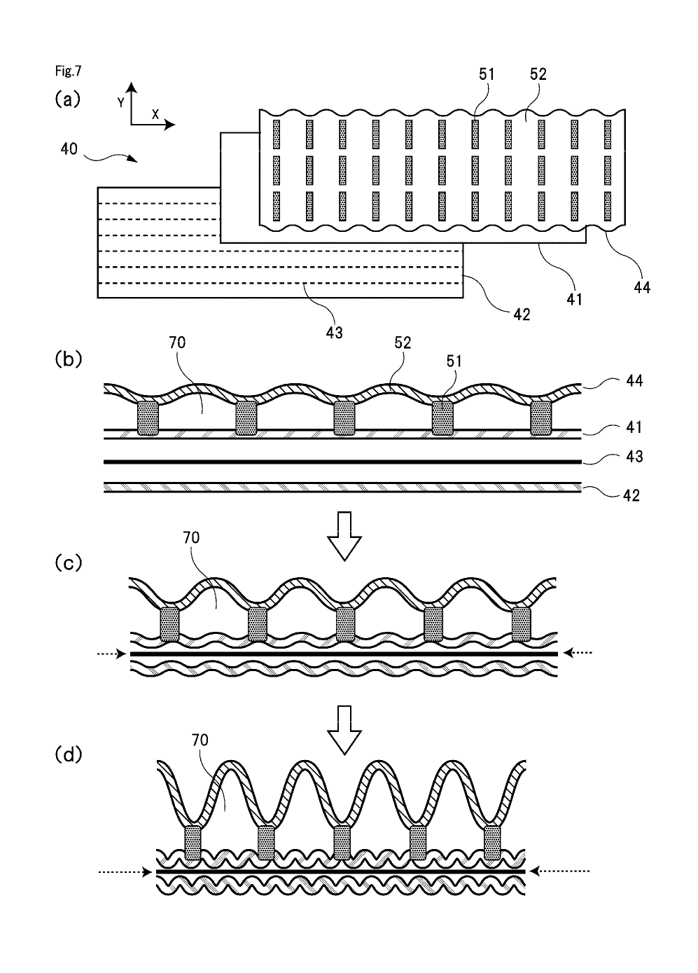

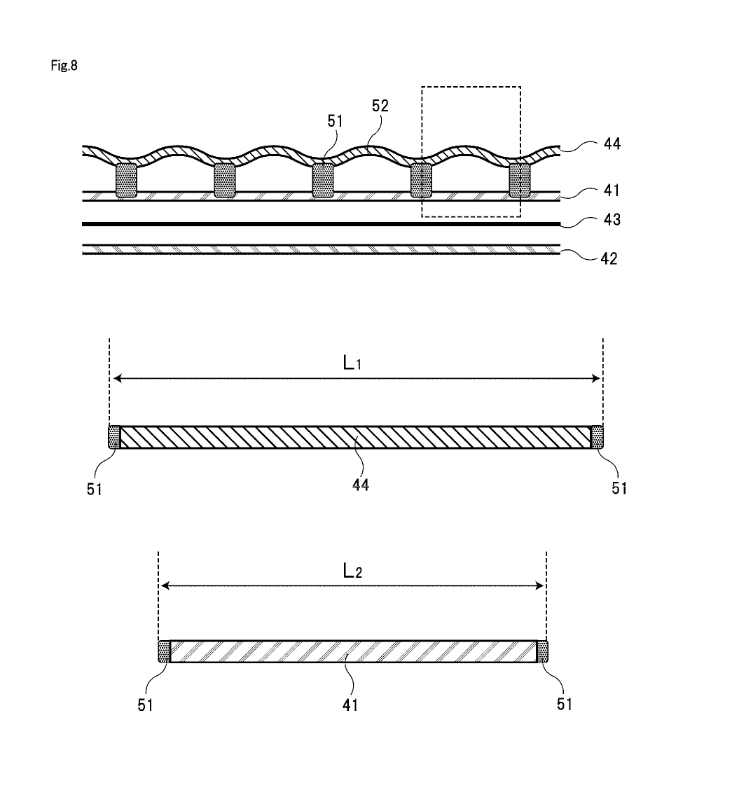

Next, a description will be given of a more preferable mode of the cushion forming sheet 44 with reference to FIGS. 7(a) to 7(d) and FIG. 8. As with FIGS. 4(a) to 4(d), FIGS. 7(a) to 7(d) are schematic views conceptually illustrating various components included in the cushion portion 40. As illustrated in FIG. 7(a), the cushion portion 40 includes the internal sheet 41, the external sheet 42, and the plurality of waist stretchable members 43, and the cushion forming sheet 44. In FIG. 7(a), the plurality of apertures 60 formed in the cushion forming sheet 44 is omitted to avoid complicating the figure. The plurality of apertures 60 may be formed in the cushion forming sheet 44 according to the above-described pattern. However, an invention illustrated in FIGS. 7(a) to 7(d) and FIG. 8 may be used as an invention in which the apertures 60 are not formed in the cushion forming sheet 44.

FIG. 7(b) illustrates a cross-sectional view in a state in which the external sheet 42, the waist stretchable members 43, the internal sheet 41, and the cushion forming sheet 44 overlap one another in order from a lower side (non-skin contact surface side) of the figure. FIG. 7(b) illustrates a state in which the waist stretchable members 43 do not contract. As illustrated in FIG. 7(b), the waist stretchable members 43 are fixed by being interposed between the internal sheet 41 and the external sheet 42 in the extended state. In addition, the cushion forming sheet 44 is joined to a skin facing surface side of the internal sheet 41 in the joining portions 51 intermittently formed in the width direction. Herein, as illustrated in FIG. 7(b), the internal sheet 41 and the external sheet 42 are in a stretched state without looseness in the width direction in accordance with the extended state of the waist stretchable members 43. In contrast, the cushion forming sheet 44 is in a slightly loose state in the width direction even when the waist stretchable members 43 are in the extended state. In this way, a characteristic of the invention is that the cushion forming sheet 44 is intermittently joined to the internal sheet 41 while being loosened.

As illustrated in FIG. 7(b), in the invention, even when the waist stretchable members 43 do not contract, and the internal sheet 41 and the external sheet 42 are in a stretched state without looseness in the width direction, the cushion forming sheet 44 is in the slightly loose state in the width direction. For this reason, even when the waist stretchable members 43 are fully stretched, the space 70 is generated between the cushion forming sheet 44 and the internal sheet 41, the cushion forming sheet 44 is in a slightly rising state.

When a structure of the cushion portion 40 of the invention illustrated in FIG. 7(b) is compared with the structure illustrated in FIG. 4(b), a difference therebetween is clear. In the structure illustrated in FIG. 4(b), each of the internal sheet 41, the external sheet 42, and the cushion forming sheet 44 is in an extended state without looseness in the width direction when the waist stretchable members 43 are in the extended state. For this reason, no wrinkle is formed in the cushion forming sheet 44 when the waist stretchable members 43 are in the extended state. For this reason, for example, when a wearer having a large abdominal circumference wears the disposable diaper, the waist stretchable members 43 do not contract, and thus no wrinkle is formed in the cushion forming sheet 44. In contrast, in the structure of the cushion portion 40 illustrated in FIG. 7(b), the cushion forming sheet 44 rises from the internal sheet 41 to form a wrinkle even when the waist stretchable members 43 are in the extended state. For this reason, for example, even when the wearer having the large abdominal circumference wears the disposable diaper, and thus contraction of the waist stretchable members 43 is small, a wrinkle is formed by the cushion forming sheet 44. Therefore, even when the waist stretchable members 43 do not contract, it is possible to sufficiently exhibit flexibility and a cushioning property around the waist of the diaper.

Next, FIG. 7(c) illustrates an appearance of the slightly contracting waist stretchable members 43, and FIG. 7(d) illustrates an appearance of the sufficiently contracting waist stretchable members 43. As illustrated in FIGS. 7(c) and 7(d), it is understood that the wrinkle formed by the cushion forming sheet 44 increases in size due to contraction of the waist stretchable members 43. Such a phenomenon is clear when FIGS. 7(c) and 7(d) are compared with FIGS. 4(c) and 4(d). That is, as illustrated in FIGS. 7(a) to 7(d), in a state in which the waist stretchable members 43 contract, the cushion forming sheet 44 of FIGS. 7(c) and 7(d) may form a obviously large wrinkle when compared to the cushion forming sheet of FIGS. 4(c) and 4(d). For this reason, when the cushion forming sheet 44 of FIGS. 7(c) and 7(d) is employed, the waist gather formed around the waist of the diaper has further enhanced flexibility and more excellent air permeability.

Herein, FIG. 8 is an explanatory drawing for a comparison between a length of the cushion forming sheet 44 and a length of the internal sheet 41 joined to the cushion forming sheet 44. In more detail, the cushion forming sheet 44 needs to be longer than the internal sheet 41 between joining portions 51 in order to generate looseness only in the cushion forming sheet 44 without generating looseness in the internal sheet 41 in the extended state of the waist stretchable members 43 as described above. As illustrated in FIG. 8, each of the cushion forming sheet 44 and the internal sheet 41 is cut between two joining portions 51 adjacent to each other in the width direction of the diaper. Then, the cut cushion forming sheet 44 and internal sheet 41 are extended in a state free of looseness. In this instance, the length of the cushion forming sheet 44 between the two joining portions 51 is set to “L₁”. In addition, the length of the internal sheet 41 between the two joining portions 51 is set to “L₂”. As illustrated in FIG. 8, when the length (L₁) is compared with the length (L₂), it is understood that the length (L₁) is longer. In this way, the cushion forming sheet 44 and the internal sheet 41 are cut between the two joining portions 51 adjacent to each other in the width direction of the diaper, and the lengths thereof are compared. In this instance, when the length of the cushion forming sheet 44 is set to be longer, a wrinkle may be formed in the cushion forming sheet 44 even when the waist stretchable members 43 do not contract.

Herein, when the length (L₂) of the internal sheet 41 is set to 100%, the length (L₁) of the cushion forming sheet 44 is preferably in a range of 103 to 300%. In addition, the length (L₁) of the cushion forming sheet 44 may be in a range of 110 to 290% or 120 to 280%, and may be in a range of 150 to 250% with respect to the length (L₂) of the internal sheet 41. In this way, when the length (L₁) is set to at least 103% or more of the length (L₂), a wrinkle may be generated in the cushion forming sheet 44 even when the waist stretchable members 43 insufficiently contract. On the other hand, when the length (L₁) exceeds 300% of the length (L₂), the cushion forming sheet 44 is greatly bent, and the diaper gives discomfort at the time of wearing. For this reason, in the invention, the length (L₁) is particularly preferably in a range of 103% or more and 300% or less of the length (L₂).

### (2-2. Embodiments of Cushion Portion)

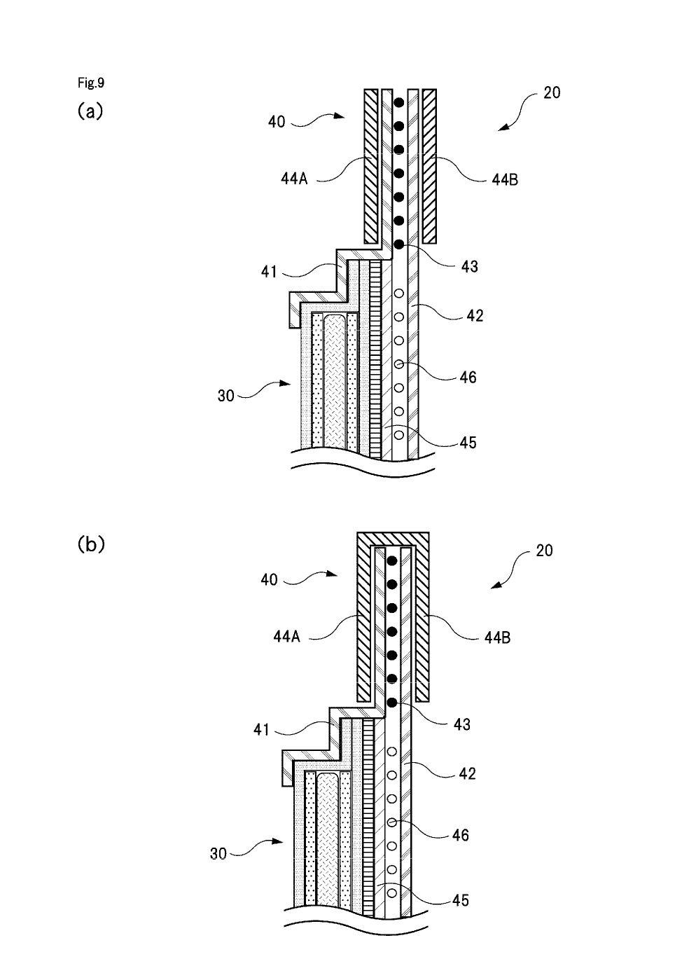

Next, a description will be given of other embodiments of the cushion portion 40 in the disposable diaper 100 of the invention with reference to FIG. 9(a) to FIG. 11(b). In the embodiments below, basically, a description will be focused on a difference from the above-described embodiment illustrated in FIG. 3.

In an embodiment illustrated in FIG. 9(a), the cushion portion 40 includes a cushion forming sheet 44A intermittently joined to the internal sheet 41 and a cushion forming sheet 44B intermittently joined to the external sheet 42. That is, the cushion forming sheet 44A on the inside is joined to a skin facing surface of the internal sheet 41. In addition, the cushion forming sheet 44B on the outside is joined to a non-skin facing surface of the external sheet 42. In this way, in the invention, the cushion forming sheets 44A and 44B may be disposed on both sides corresponding to the inside and the outside.

In an embodiment illustrated in FIG. 9(b), the cushion forming sheet 44A on the inside and the cushion forming sheet 44B on the outside are configured by one sheet member. In more detail, the one sheet member is folded back at an end edge of the disposable diaper in the longitudinal direction, thereby setting a portion positioned on the inside of a return line to the cushion forming sheet 44A on the inside, and setting a portion positioned on the outside of the return line to the cushion forming sheet 44B on the outside. In this way, in the invention, the cushion forming sheet 44A on the inside and the cushion forming sheet 44B on the outside may be configured by the one sheet member.

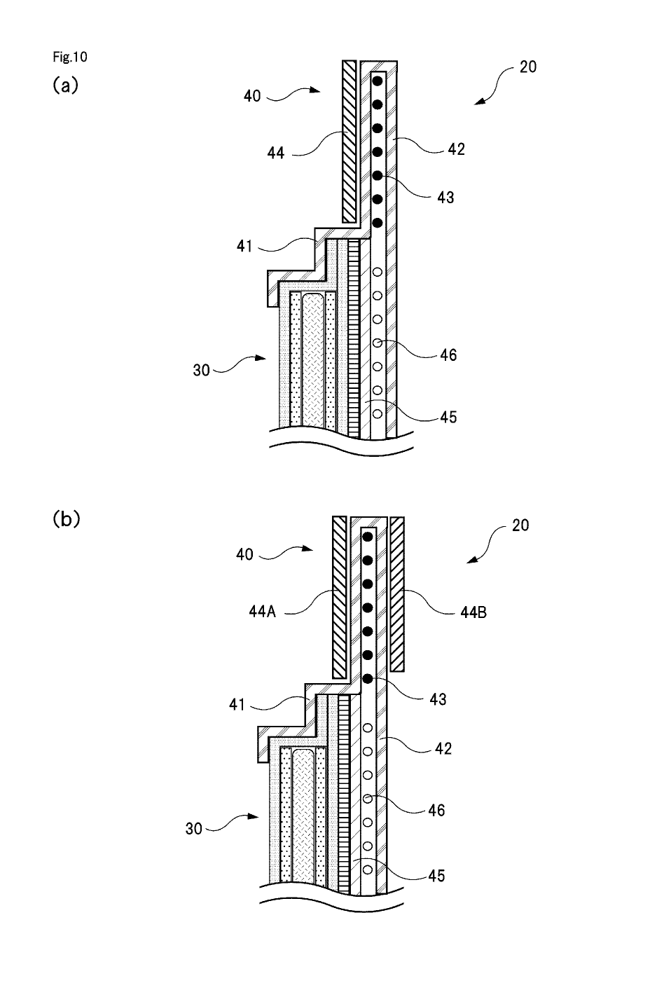

In an embodiment illustrated in FIG. 10(a), the internal sheet 41 and the external sheet 42 are configured by one sheet member. In more detail, the one sheet member is folded back at an end edge of the disposable diaper in the longitudinal direction, and the plurality of waist stretchable members 43 is fixed by being interposed therebetween. Then, a portion positioned on the inside of a return line is set to the internal sheet 41, and a portion positioned on the outside of the return line is set to the external sheet 42. In this way, in the invention, the internal sheet 41 and the external sheet 42 may be configured by the one sheet member.

In an embodiment illustrated in FIG. 10(b), the internal sheet 41 and the external sheet 42 are configured by one sheet member, and a cushion forming sheet 44A on the inside and a cushion forming sheet 44B on the outside formed using different sheet members are provided. In more detail, the cushion forming sheet 44A on the inside is joined to the skin facing surface of the internal sheet 41, and the cushion forming sheet 44B on the outside is joined to the non-skin facing surface of the external sheet 42. In this way, in the invention, the internal sheet 41 and the external sheet 42 may be configured by the one sheet member, and both the cushion forming sheet 44A on the inside and the cushion forming sheet 44B on the outside may be provided.

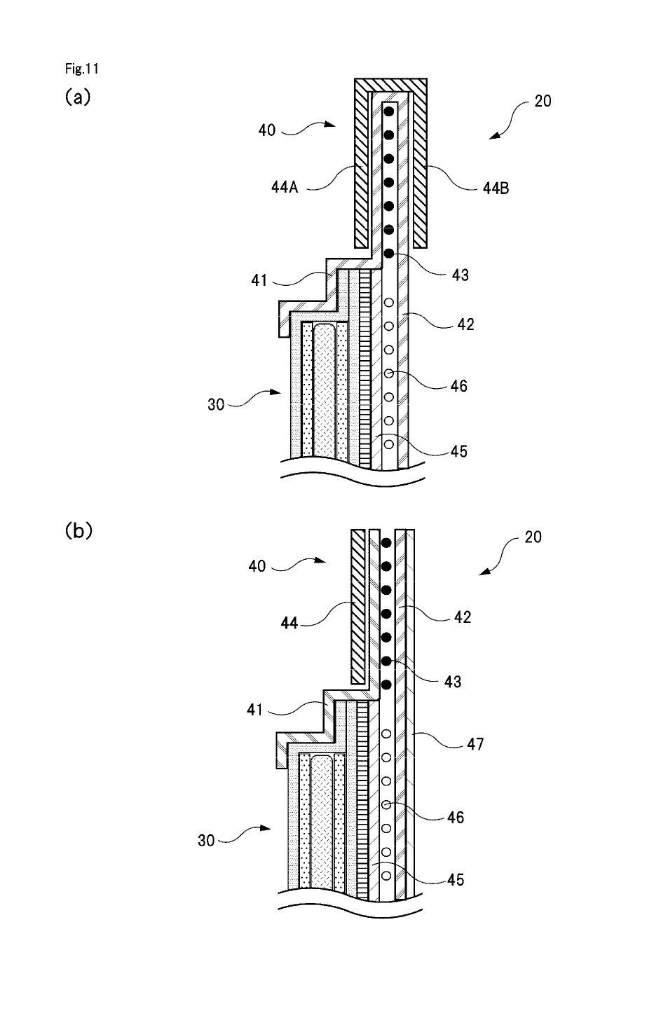

In an embodiment illustrated in FIG. 11(a), the internal sheet 41 and the external sheet 42 are configured by one sheet member, and a cushion forming sheet 44A on the inside and a cushion forming sheet 44B on the outside are configured by one sheet member. In more detail, the one sheet member is folded back at an end edge of the disposable diaper in the longitudinal direction, thereby setting a portion positioned on the inside of a return line to the cushion forming sheet 44A on the inside, and setting a portion positioned on the outside of the return line to the cushion forming sheet 44B on the outside. In this way, in the invention, the internal sheet 41 and the external sheet 42 are configured by the one sheet member, and the cushion forming sheet 44A on the inside and the cushion forming sheet 44B on the outside may be configured by the one sheet member.

In an embodiment illustrated in FIG. 11(b), an outermost layer sheet 47 is bonded to the non-skin facing surface side of the external sheet 42. Unlike the cushion forming sheet 44B on the outside described above, this outermost layer sheet 47 is not intermittently joined to the external sheet 42. For example, the outermost layer sheet 47 is fully bonded thereto using a hot-melt adhesive, etc. The outermost layer sheet 47 is a sheet member for reinforcing the external sheet 42 and improving a touch thereof. In this way, the outermost layer sheet may be bonded to the non-skin facing surface of the external sheet 42.

### (2-3. Absorbent Main Body)

As illustrated in FIG. 2 and FIG. 3, the absorbent main body 30 is held while being bridged between the front body part outer packaging body 10 and the rear body part outer packaging body 20, is positioned in the crotch portion of the wearer at the time of wearing the disposable diaper 100, and absorbs and holds a liquid such as urine excreted by the wearer. The absorbent main body 30 is disposed across the front body part 1 and the rear body part 2 around the crotch portion 3 of the disposable diaper. As illustrated in FIG. 2 and FIG. 3, the absorbent main body 30 includes an absorber 31, a top sheet 32, a back sheet 33, a cover sheet 34, and a pair of solid gathers 35.

The absorber 31 is a member for absorbing a liquid such as urine, and holding the absorbed liquid. The absorber 31 is disposed between the liquid-permeable top sheet 32 and the liquid-impermeable back sheet 33. The absorber 31 has a function of absorbing a liquid penetrating the top sheet 32, and is made of an absorbent material. A known material may be employed as the absorbent material contained in the absorber 31. For example, a flap pulp, a super absorbent polymer, or a hydrophilic sheet may be used as the absorbent material. The absorber 31 may be enclosed by a core wrap sheet 36. Tissue paper, absorbent paper, a nonwoven fabric subjected to a hydrophilic treatment, etc. may be appropriately used as the core wrap sheet 36.

The top sheet 32 is a member that directly comes into contact with a skin of the crotch portion of the wearer, and allows a liquid such as urine to penetrate to the absorber 31. For this reason, the top sheet 32 is made of a liquid-permeable material having high flexibility. In addition, the top sheet 32 is disposed to cover a skin contact surface side of the absorber 31. Examples of the liquid-permeable material contained in the top sheet 32 include a fabric, a nonwoven fabric, or a porous film. In addition, for example, a material obtained by performing a hydrophilic treatment on a fiber of a thermoplastic resin such as polypropylene, polyethylene, polyester, or nylon, and then forming a nonwoven fabric using the fiber may be used as the top sheet 32.

The back sheet 33 is a member for preventing the liquid, which penetrates the top sheet 32 and is absorbed by the absorber 31, from leaking to the outside of the diaper. For this reason, the back sheet 33 is made of a liquid-impermeable material. The back sheet 33 covers the absorber 31 from the non-skin contact surface side to prevent a liquid leakage from a bottom surface of the absorber 31. An example of the impermeable material contained in the back sheet 33 corresponds to a liquid-impermeable film made of polyethylene resin. In particular, it is preferable to use a microporous polyethylene film in which a plurality of minute holes having a size in a range of 0.1 to 4 μm is formed.

The cover sheet 34 is a member for reinforcing the back sheet 33 and improving a touch thereof. The cover sheet 34 is bonded to the non-skin contact surface side of the back sheet 33. A woven fabric or a nonwoven fabric is used as a material contained in the cover sheet 34. In particular, it is preferable to use a nonwoven fabric or a wet-laid nonwoven fabric made of a thermoplastic resin such as polyethylene, polypropylene, or polyester as the material contained in the cover sheet 34.

The pair of solid gathers 35 is a member that rises along both side edges of the absorber 31, and prevents a side leakage of urine excreted by the wearer. A solid gather stretchable material is disposed at a distal end of each of the solid gathers 35, and rises in a skin contact direction of the wearer when the stretchable material contracts. For this reason, the pair of solid gathers 35 serves as a urine leakproof wall, and prevents a side leakage in which urine penetrating the top sheet 32 or urine failing to be absorbed by the absorber 31 leaks from an opening around a leg portion of the disposable diaper. The solid gathers 35 may employ a known configuration used for a conventional disposable diaper. For example, the solid gathers 35 may be formed by interposing and fixing the solid gather stretchable material in an extended state between layers of a water-repellent sheet. For example, a nonwoven fabric sheet obtained using a manufacturing method such as card embossing, spunbond, etc. may be used as the water-repellent sheet. In particular, it is preferable to use a nonwoven fabric such as SMS, SMMS, etc. having a high waterproofing property.

Hereinbefore, in this specification, a description has been given of the embodiments of the invention with reference to the drawings to express content of the invention. However, the invention is not restricted to the above embodiments, and includes a modified mode or an improved mode clear to those skilled in the art based on information described in this specification.

For example, in the invention, a cushioning property may be assigned by manufacturing the cushion sheet illustrated in FIGS. 4(a) to 4(d) and FIGS. 7(a) to 7(d) as a separate body from the disposable diaper, and bonding the cushion sheet around the waist of the disposable diaper.

In more detail, as illustrated in FIG. 4(a), the cushion sheet is a sheet-shaped member in which one or a plurality of elastic stretchable members is fixed in parallel between a plurality of sheet members. The cushion sheet includes the internal sheet 41, the external sheet 42, the one or the plurality of elastic stretchable members 43, and the cushion forming sheet 44. The one or the plurality of elastic stretchable members 43 is fixed by being interposed between the internal sheet 41 and the external sheet 42 in an extended state in a certain direction (X direction). The cushion forming sheet 44 overlaps both or any one of the internal sheet 41 and the external sheet 42, and is intermittently joined thereto such that the joining portions 51 and the non-joining portions 52 are alternately continued in the certain direction (X direction). In addition, the cushion forming sheet 44 includes a sheet member in which the plurality of apertures 60 or cavities are regularly formed. The plurality of apertures 60 or cavities are grouped into the row group (R) and the column group (C). The row group (R) refers to a group formed by connecting apertures 60 or cavities adjacent in a certain direction (X direction). The column group (C) refers to a group formed by connecting apertures 60 or cavities adjacent in a direction (Y direction) perpendicular to the certain direction. Herein, the connection straight line (A) connecting the plurality of apertures 60 or cavities within the column group (C) is inclined at the predetermined angle (θ) with respect to the reference straight line (F) extending in the perpendicular direction (Y direction). The predetermined angle (θ) is preferable in the range of 5 to 45 degrees.

In addition, as illustrated in FIGS. 7(a) to 7(d) and FIG. 8, the length (L₁) of the cushion forming sheet 44 between the joining portions 51 in the certain direction is preferable longer than the length (L₂) of the internal sheet 41 or the external sheet 42 to which the cushion forming sheet 44 is joined in the certain direction between the joining portions 51. In this case, the above-described plurality of apertures 60 or cavities are preferably formed in the cushion forming sheet 44. However, in the mode illustrated in FIGS. 7(a) to 7(d) and FIG. 8, the apertures 60 or cavities may not be formed in the cushion forming sheet 44.

As described in the foregoing, a cushioning property may be easily assigned to an existing disposable diaper by manufacturing a cushion sheet separately from the disposable diaper. For this reason, manufacturing of only the cushion sheet is a preferable mode of the invention.

### INDUSTRIAL APPLICABILITY

The invention is preferably applicable to a disposable diaper manufacturing industry.

### REFERENCE SIGNS LIST

1 . . . front body part
2 . . . rear body part
3 . . . crotch portion
4 . . . waist opening
5 . . . leg opening
10 . . . front body part outer packaging body
20 . . . rear body part outer packaging body
30 . . . absorbent main body
31 . . . absorber
32 . . . top sheet
33 . . . back sheet
34 . . . cover sheet
35 . . . solid gather
36 . . . core wrap sheet
40 . . . cushion portion
41 . . . internal sheet
42 . . . external sheet
43 . . . waist stretchable member
44 . . . cushion forming sheet
44a . . . intermittent joining region
44a . . . full joining region
45 . . . inner sheet
46 . . . stretchable tammy member
47 . . . outermost layer sheet
51 . . . joining portion
52 . . . non-joining portion
60 . . . apertures
61 . . . first aperture
62 . . . second aperture
70 . . . ventilation passage (space)

1. A disposable diaper divided into a front body part (1), a rear body part (2), and a crotch portion (3) positioned between the front body part (1) and the rear body part (2) in a longitudinal direction (Y direction), a cushion portion (40) being formed in both or any one of the front body part (1) and the rear body part (2),
wherein the cushion portion (40) includes:
an internal sheet (41);
an external sheet (42) positioned on a side of a non-skin facing surface of the internal sheet (41);
one or a plurality of waist stretchable members (43) fixed by being interposed between the internal sheet (41) and the external sheet (42) while extending in a width direction (X direction) of the disposable diaper; and
a cushion forming sheet (44) overlapping both or any one of the internal sheet (41) and the external sheet (42), the cushion forming sheet (44) being intermittently joined thereto such that joining portions (51) and non-joining portions (52) are alternately continued in the width direction,
the cushion forming sheet (44) includes a sheet member in which a plurality of apertures (60) or cavities are regularly formed,
the plurality of apertures (60) or cavities are grouped into
a row group (R) formed by connecting apertures (60) or cavities adjacent to each other in the width direction, and
a column group (C) formed by connecting apertures (60) or cavities adjacent to each other in the longitudinal direction, and
a connection straight line (A) connecting a plurality of apertures (60) or cavities within the column group (C) is inclined at a predetermined angle (θ) with respect to a reference straight line (F) extending in the longitudinal direction.

2. The disposable diaper according to claim 1, wherein the predetermined angle (θ) is 5 degrees or more and 45 degrees or less.

3. The disposable diaper according to claim 1, wherein, when the reference straight line (F) is drawn to pass through a center of a first aperture (61) and a center of a second aperture (62), an interval (P) between the first aperture (61) and the second aperture (62) in the longitudinal direction is three times or more an interval (D) in the longitudinal direction between adjacent apertures (60) included in the column group (C).

4. The disposable diaper according to claim 1, wherein a length (L₁) of the cushion forming sheet (44) in the width direction between the joining portions (51) is longer than a length (L₂) of the internal sheet (41) or the external sheet (42) to which the cushion forming sheet (44) is joined in the width direction between the joining portions (51).

5. A cushion sheet in which one or a plurality of elastic stretchable members is fixed between a plurality of sheet members, the cushion sheet comprising:
an internal sheet (41);
an external sheet (42);
one or a plurality of elastic stretchable members (43) fixed by being interposed between the internal sheet (41) and the external sheet (42) while extending in a certain direction (X direction); and
a cushion forming sheet (44) overlapping both or any one of the internal sheet (41) and the external sheet (42), the cushion forming sheet (44) being intermittently joined thereto such that joining portions (51) and non-joining portions (52) are alternately continued in the certain direction,
wherein the cushion forming sheet (44) includes a sheet member in which a plurality of apertures (60) or cavities are regularly formed,
the plurality of apertures (60) or cavities are grouped into
a row group (R) formed by connecting apertures (60) or cavities adjacent to each other in the certain direction, and
a column group (C) formed by connecting apertures (60) or cavities adjacent to each other in a perpendicular direction (Y direction) perpendicular to the certain direction, and
a connection straight line (A) connecting a plurality of apertures (60) or cavities within the column group (C) is inclined at a predetermined angle (θ) with respect to a reference straight line (F) extending in the perpendicular direction.
